(19) United States
(12) Patent Application Publication
(10) Pub. No.: US 2025/0137974 A1
(43) Pub. Date: May 1, 2025

(54) IMPLANTABLE WIRELESS SENSOR APPARATUS AND ULTRASONIC ACTUATOR THEREOF

(71) Applicant: KOC UNIVERSITESI, Istanbul (TR)

(72) Inventor: Levent BEKER, Istanbul (TR)

(73) Assignee: KOC UNIVERSITESI, Istanbul (TR)

(21) Appl. No.: 18/682,935

(22) PCT Filed: Aug. 12, 2021
(86) PCT No.: PCT/TR2021/050796

§ 371 Date: Feb. 12, 2024

(51) Int. Cl. G01N 29/24; G01N 29/34

(52) U.S. Cl. CPC G01N 29/245; G01N 29/2406; G01N 29/2481; G01N 29/348; G01N 2291/014; G01N 2291/02483; G01N 2291/106

(57) ABSTRACT

An implantable wireless sensor apparatus comprises a transducer unit having at least one ultrasonic transducer, and at least one body sensor coupled to the transducer unit, wherein electrical resonance frequency of the apparatus (Fₑ) is within the bandwidth (BW) of mechanical resonance frequency of the ultrasonic unit (Fₘ).

### FIELD OF THE INVENTION

The present invention relates to a battery-free implantable wireless sensor apparatus capable of ultrasonic communication, an ultrasonic actuator for the apparatus and a method for processing the data received from the apparatus.

### BACKGROUND OF THE INVENTION

Body sensor implants refer to a variety of biomedical devices operating within human/animal body for monitoring various health parameter. In recent days, recovery phases of a postoperative tissue are frequently observed wirelessly via body sensor implants.

The implants in the state of the art comprises a sensor for receiving a health parameters, electronic components including a communication unit for wirelessly transferring data of the sensor and a battery for powering the implant. Data of a health parameter measured by the sensor is transferred to outside of the body by the communication unit having active electronic components via a short-range wireless technology such as Bluetooth and NFC. Having active electronic components and battery makes the implants bulky and reduces the service life of the implants due to the limited lifespan of the batteries. To reduce this cumbersome, some implants have a capable of inductive charging. However, the components for inductive charging such as capacitors and antennas add another bulkiness to the implant and these implants cannot be placed to deep tissues such as brain, kidney and heart since the necessary magnetic field for penetrating the delicate tissue and charging the implant is not safe. Moreover, inductive charging increases the heat in the tissue surrounding the implant which can cause a detrimental effect on the tissue.

In another type of the body sensor implants, ultrasonic waves are used to read the sensor data. These implants comprise an integrated circuit such as application specific integrated circuit (ASIC) and an ultrasonic transducer. A communication and power transmission between an interrogator and the implant are set by a pulse-echo method based on transmitting and receiving ultrasonic waves. The interrogator transmits ultrasonic waves to the implant. The ultrasonic transducers of the implant transform ultrasonic acoustic waves into electrical signals and powers the integrated circuit. The integrated circuit is coupled to sensor or electrodes which are engaged to a tissue. A signal processing unit and a modulation circuit embedded in the integrated circuit which are adapted to modulate the electrode signals and transmit the modulated signal. Then, the modulated signal received from the interrogator is demodulated and the data of the electrodes is acquired. One disadvantage of these implants is that they generally use an amplitude modulation (AM). Since AM signals are susceptible for intracorporeal signal loss, angle of the measurement, tissue/skin thickness, the measurements are neither reliable and nor practical. Although some of these implants does not requires a battery, active electronic components need more energy than a passive ones which requires to increase the number of ultrasonic transducers to power the active electronic components. On the other hand, due to the inherent characteristic of the active electronic components and batteries, implants cannot be made as biodegradable which may requires an implant removal surgery.

Since the mentioned drawbacks of the known body sensor implants, there is need for an implantable wireless sensor apparatus without an active electronic components and battery in the state of the art.

### BRIEF DESCRIPTION OF THE DRAWINGS

An exemplary embodiment of the present invention is illustrated by way of example in the accompanying drawings to be more easily understood and uses thereof will be more clear when considered in view of the detailed description, in which like reference numbers indicate the same or similar elements, and the following figures in which:

FIG. 1 is a schematic view of an implantable wireless sensor apparatus and an ultrasonic actuator in one exemplary embodiment of the present invention.

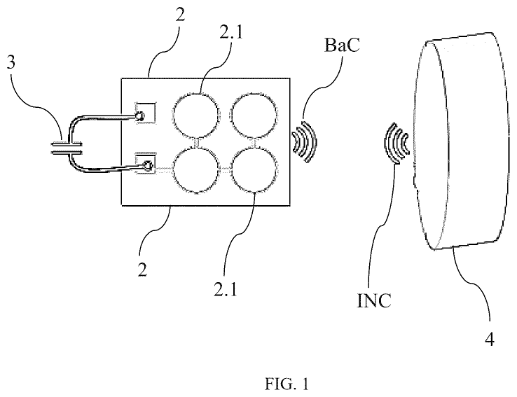

FIG. 2 is a schematic view of two adjacent implantable wireless sensor apparatus having different transmitting channels and an ultrasonic actuator in one exemplary embodiment of the present invention.

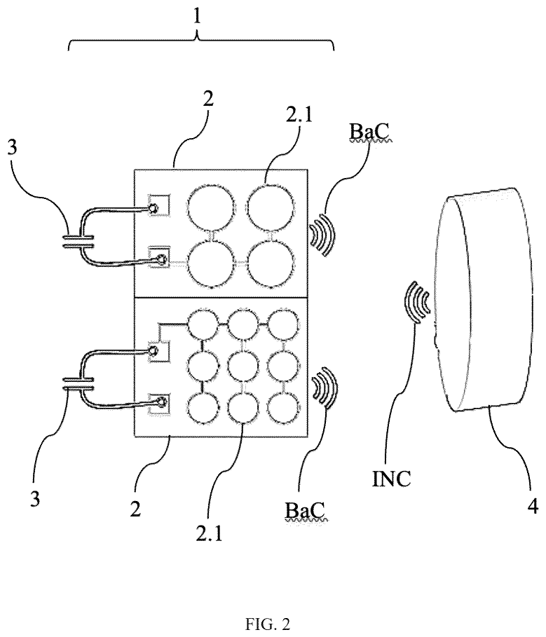

FIG. 3 is an overlapping graph showing that electrical resonance frequency of the apparatus (Fₑ) is within the bandwidth (BW) of mechanical resonance frequency of the ultrasonic unit (Fₘ) in one exemplary embodiment of the present invention.

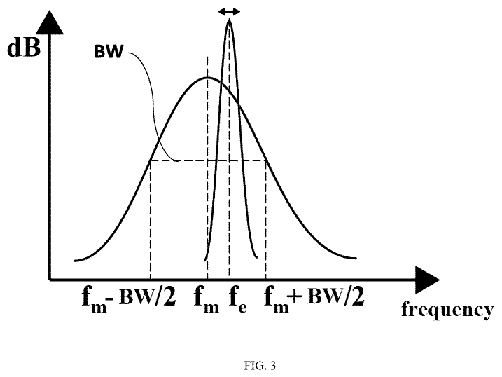

FIG. 4 is a graph showing backscattered ultrasonic waves and ring-down part thereof in one exemplary embodiment of the present invention.

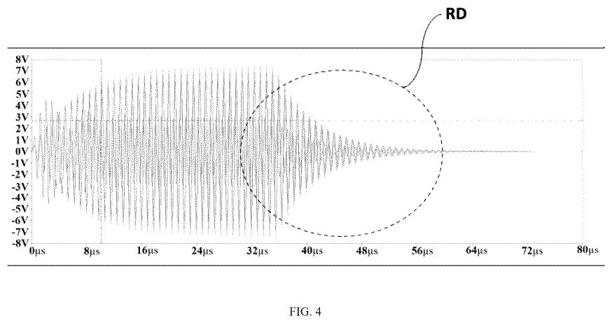

The elements illustrated in the figures are numbered as follows:

1. Wireless sensor apparatus

2. Transducer unit

2.1. ultrasonic transducer

3. Body sensor

4. Ultrasonic actuator

Fₑ. Electrical resonance frequency of the apparatus

Fₘ. Mechanical resonance frequency of the ultrasonic unit

BW. Bandwidth

INC. Incident ultrasonic waves

RD. Ring-down part

BaC. Backscattered ultrasonic waves

### DETAILED DESCRIPTION

Embodiments of the present invention relates to an implantable wireless sensor apparatus (1) comprising a transducer unit (2) having at least one ultrasonic transducer (2.1). The apparatus has at least one body sensor (3) which is coupled to the transducer unit (2). The transducer unit (2) is for transforming ultrasonic waves into electrical signals powering/exciting the body sensor (3) and for transforming the electrical signals into ultrasonic waves carrying data of the powered body sensor (3). In all embodiment of the present invention, electrical resonance frequency of the apparatus (Fₑ) is within the bandwidth (BW) of mechanical resonance frequency of the ultrasonic unit (Fₘ).

The invention works as follows:

After implantation of the apparatus to a human/animal body, ultrasonic waves (incident ultrasonic waves (INC)) are sent towards the apparatus from the outside of the body. The incident ultrasonic waves (INC) are received by the transducer unit (2). The ultrasonic transducers (2.1) transforms the ultrasonic waves into electrical signals at an electrical resonance frequency of the apparatus (Fₑ). The transducer unit (2) has a particular mechanical resonance frequency bandwidth (BW) which mainly depends on several physical parameters of the ultrasonic transducers (2.1). The apparatus (at least the combination of transducer unit (2) and the body sensors (3)) also has a particular electrical resonance frequency which depends on inductance, resistance and capacitance of the apparatus. The electrical signals at an electrical resonance frequency of the apparatus (Fₑ) power/excite the body sensor (3). Changes in inductance, resistance and/or capacitance of the body sensor (3), which is directly related to the sensor measurements, change electrical resonance frequency of the apparatus (Fₑ). These changes in the electrical resonance frequency of the apparatus (Fₑ) affect and change the electrical signals. Thus, the data of the body sensor (3) is passively embedded into changed electrical signals. The changed electrical signals are transformed into backscattered ultrasonic waves (BaC) by coupled the transducer unit (2) and transferred to the outside of the body. A ring-down part (RD) of the backscattered ultrasonic waves (BaC) is a section that converges to the changed electrical signals and it directly gives information about the changed electrical signals and inherently the changed electrical resonance frequency of the apparatus (Fₑ). Ring-down profile follows e^{-D*F*t} form (D: damping ratio, F: changed electrical resonance frequency of the apparatus (Fe), t: time). By using a signal processing method comprising a Fourier Transformation, changed electrical signals are acquired which give the changed electrical resonance frequency of the apparatus (Fₑ) which is directly related to the body sensor (3) data. Thus, the body sensor (3) data is wirelessly acquired from the ring-down part (RD) of the backscattered ultrasonic waves (BaC).

One critical part of the invention is that the electrical resonance frequency of the apparatus (Fe) must be within the bandwidth (BW) of mechanical resonance frequency of the ultrasonic unit (Fₘ). Otherwise, the changed electrical resonance frequency of the apparatus (Fₑ) from the ring-down part (RD) of the backscattered ultrasonic waves (BaC) cannot be acquired due to the high noise and signal loss arising from transition between mechanical domain (ultrasonic waves) to electrical domain (electrical signal). In one embodiment of the invention, electrical resonance frequency of the apparatus (Fₑ) is determined by also considering body sensor (3) state during a maximum and minimum level of measuring interval i.e. sensor changes.

The embodiments of the invention does not require an active electronic component and a power storage unit such as a battery or capacitor/condenser which allows the wireless sensor apparatus (1) can be made in a very compact form. Thus, the sensor apparatus is usable for delicate tissues, especially deep tissue such as cardiovascular system, brain tissue or a peripheral nerves. Moreover, since the apparatus makes use of frequency (rather than amplitude) of ultrasonic waves, a reliable result (the body sensor (3) data) is acquired. Another advantage of the invention is that the measurement of the body sensor (3) is independent from the frequency of the incident ultrasonic waves (INC). Inherently, it is enough that the incident ultrasonic waves (INC) are in the bandwidth (BW) of mechanical resonance frequency of the ultrasonic unit (Fₘ).

In one embodiment of the invention, ultrasonic transducer (2.1) is a diaphragm type ultrasonic transducer (2.1). In one alternative of this embodiment, the diaphragm type ultrasonic transducer (2.1) is a piezoelectric micro-machined ultrasonic transducer (PMUT). Bandwidth (BW) of mechanical resonance frequency of the piezoelectric ceramic ultrasonic transducer (2.1) is defined by impedance parameters including nominal capacitance and/or resistance and/or inductance of the ultrasonic transducer (2.1). The impedance parameters and so the bandwidth (BW) of mechanical resonance frequency the diaphragm type ultrasonic transducers (2.1) may be adjusted by changing the mechanical features such as thickness, diameter, material properties of the diaphragm. Thus, for this embodiment, diaphragm surface area and/or diaphragm thickness of the ultrasonic transducers and/or nominal capacitance of the sensors (3) are preferably arranged such that electrical resonance frequency of the apparatus (Fₑ) is in the bandwidth (BW) of mechanical resonance frequency of the ultrasonic unit (Fₘ).

In one embodiment of the invention, the ultrasonic transducer (2.1) is a piezoelectric ceramic ultrasonic transducer (2.1). Such piezoelectric ceramic ultrasonic transducer (2.1) may be, but not limited to, miniaturized bulk piezoelectric ceramic ultrasonic transducer (2.1). Bandwidth (BW) of mechanical resonance frequency of the piezoelectric ceramic ultrasonic transducer (2.1) is defined by impedance parameters including nominal capacitance and/or resistance and/or inductance of the ultrasonic transducer (2.1). The impedance parameters and so the bandwidth (BW) of mechanical resonance frequency of the piezoelectric ceramic ultrasonic transducer (2.1) may be adjusted by changing the number of ceramics, ceramic surface area and/or ceramic thickness of the ultrasonic transducers. Thus, the number of ceramics, ceramic surface area and/or ceramic thickness of the ultrasonic transducers and/or nominal capacitance of the sensors (3) may be arranged such that electrical resonance frequency of the apparatus (Fₑ) is in the bandwidth (BW) of mechanical resonance frequency of the ultrasonic unit (Fₘ).

In another embodiment of the invention, the body sensor (3) is a capacitive, inductive or resistive sensor. The body sensor (3) may be, but not limited to, detect and/or measure proximity, pressure, position and displacement, force, fluid level in the purpose of health monitoring.

In one embodiment of the invention, the transducer unit (2) and/or the body sensor (3) are made of biodegradable materials. In one alternative of this embodiment, the biodegradable materials are coated with a triggered biodegradation layer.

In one embodiment of the invention, ultrasonic transducers (2.1) are in the form of an ultrasonic transducer array. Thus, the incident ultrasonic waves (INC) are effectively transformed into electrical signals.

In one embodiment of the invention, implantable wireless sensor apparatus (1) comprises at least two implantable wireless sensor apparatus (1) such as two adjacent implantable wireless sensor apparatus (1). In this embodiment, each transducer unit (2) has a different mechanical resonance frequency bandwidth (BW). In this way, a different transmitting channel is provided to each, preferably different type of, body sensors (3) which are coupled to its corresponding transducer unit (2).

One embodiment of the invention which is applicable for all embodiment relates to an ultrasonic actuator (4) in order for sending ultrasonic waves (the incident ultrasonic waves (INC)) to an implantable wireless sensor apparatus (1) and receiving ultrasonic waves (the backscattered ultrasonic waves (BaC)) from an implantable wireless sensor apparatus (1). The ultrasonic actuator (4) is adapted to send ultrasonic waves within the mechanical resonance frequency of the ultrasonic unit (Fₘ).

In one embodiment of the invention, the ultrasonic actuator (4) is connected to a signal generator and a processing unit with lock-in measurement to analyze backscattered waves (BaC) in order for acquiring the body sensor (3) data and generate actuation signals accordingly. The ultrasonic actuator (4), the signal generator and the signal processing unit can be configured to work as an adaptive system. For instance in one embodiment of the invention, they can be used as a pulse-echo type system to access the electrical resonance frequency of the apparatus (Fₑ). Similarly, a frequency modulated signal can be used as the actuation signal for excitation of the ultrasonic actuator (4) to produce incident ultrasonic waves (INC) such as chirp signals. In this case, the chirp signal should include electrical resonance frequency of the apparatus (Fₑ).

1. An implantable wireless sensor apparatus (1) comprising a transducer unit (2) having at least one ultrasonic transducer (2.1); and at least one body sensor (3) coupled to the transducer unit (2) characterized in that electrical resonance frequency of the apparatus (Fₑ) is within the bandwidth (BW) of mechanical resonance frequency of the ultrasonic unit (Fₘ).

2. An implantable wireless sensor apparatus (1) according to any of the preceding claims wherein the ultrasonic transducer (2.1) is a diaphragm type ultrasonic transducer (2.1).

3. An implantable wireless sensor apparatus (1) according to claim 2, wherein the diaphragm type ultrasonic transducer (2.1) is a piezoelectric micro-machined ultrasonic transducer (PMUT).

4. An implantable wireless sensor apparatus (1) according to claim 2 or 3, wherein diaphragm surface area and/or diaphragm thickness of the ultrasonic transducers and/or nominal capacitance of the sensors (3) are arranged such that electrical resonance frequency of the apparatus (Fₑ) is in the bandwidth (BW) of mechanical resonance frequency of the ultrasonic unit (Fₘ).

5. An implantable wireless sensor apparatus (1) according to claim 1 wherein the ultrasonic transducer (2.1) is a piezoelectric ceramic ultrasonic transducer (2.1).

6. An implantable wireless sensor apparatus (1) according to claim 5, wherein number of ceramics, ceramic surface area and/or ceramic thickness of the ultrasonic transducers and/or nominal capacitance of the sensors (3) are arranged such that electrical resonance frequency of the apparatus (Fₑ) is in the bandwidth (BW) of mechanical resonance frequency of the ultrasonic unit (Fₘ).

7. An implantable wireless sensor apparatus (1) according to any of the preceding claims, wherein the body sensor (3) is a capacitive, inductive or resistive sensor.

8. An implantable wireless sensor apparatus (1) according to any of the preceding claims, wherein the transducer unit (2) and/or the body sensor (3) are made of biodegradable materials.

9. An implantable wireless sensor apparatus (1) according claim 8, comprising a triggered biodegradation layer which is coated on the biodegradable materials in order to initiate biodegrading of the materials by a controlled effect.

10. An implantable wireless sensor apparatus (1) according to any of the preceding claims wherein ultrasonic transducers (2.1) are in the form of an ultrasonic transducer array.

11. An implantable wireless sensor apparatus (1) comprising at least two implantable wireless sensor apparatus (1) according to any of the preceding claims wherein each transducer unit (2) has a different mechanical resonance frequency bandwidth (BW) in order to provide a different transmitting channel to each body sensors (3) coupled to the corresponding transducer unit (2).

12. An ultrasonic actuator (4) in order for sending ultrasonic waves to and receiving ultrasonic waves from an implantable wireless sensor apparatus (1) according to any of the preceding claims.
